(19) United States
(12) Patent Application Publication
(10) Pub. No.: US 2023/0346645 A1
(43) Pub. Date: Nov. 2, 2023

(54) METHOD OF TITRATING DOSE OF PSYCHEDELICS

(71) Applicants: Robert Barrow, Madison, WI (US); Daniel R. Karlin, New York, NY (US)

(72) Inventors: Robert Barrow, Madison, WI (US); Daniel R. Karlin, New York, NY (US)

(73) Assignee: Mind Medicine, Inc., New York, NY (US)

(21) Appl. No.: 17/734,098

(22) Filed: May 1, 2022

(51) Int. Cl. A61J 7/00; A61J 1/03; G16H 20/10; A61J 7/04; A61K 45/06

(52) U.S. Cl. CPC A61J 7/0076; A61J 1/035; G16H 20/10; A61J 7/04; A61K 45/06

(57) ABSTRACT

A method of dosing a psychedelic that avoids the side effects of hallucinations and perceptual disturbances by administering the psychedelic to an individual in a titrating dosing regimen and reducing side effects of hallucinations and perceptual disturbances. A kit for administering a titrating dosing regimen of a psychedelic, including a pharmaceutically effective amount of the psychedelic in dosage forms separated in packaging according to dose and time of administration in a titrating dosing regimen, and instructions for use. A method of treating an individual with psychedelics, by administering the psychedelic to the individual having a condition or disease in a titrating dosing regimen and reducing side effects of hallucinations and perceptual disturbances during treatment.

### BACKGROUND OF THE INVENTION

### 1. Technical Field

The present invention relates to compositions and methods for dosing psychedelics.

### 2. Background Art

Psychedelics including lysergic acid diethylamide (LSD) are substances capable of inducing unique subjective effects including alterations of consciousness, positive emotions, enhanced introspection, changes in the perception of the environment, the body, and the self as well as synesthesia, mystical-type experiences, and experiences of ego dissolution (Carhart-Harris et al., 2016b; Dolder et al., 2016; Holze et al., 2021; Liechti, 2017; Passie et al., 2008; Schmid et al., 2015).

All serotonergic psychedelics including LSD, psilocybin, DMT, and mescaline are nonspecific serotonin agonists including agonist activity at the serotonin 5-HT2A receptor (Rickli et al., 2016) and may therefore produce overall largely similar effects. Additionally, psychedelic substances produce their acute effects in humans via activation of the serotonin 5-HT2A receptor as specifically shown in clinical studies for LSD (Holze et al., 2021; Preller et al., 2017). LSD and other hallucinogens are partial agonists of the serotonin 5-HT2A receptor (López-Giménez, et al. Hallucinogens and Serotonin 5-HT2A Receptor-Mediated Signaling Pathways. Curr Top Behav Neurosci. 2018;36:45-73; Canal CE. Serotonergic Psychedelics: Experimental Approaches for Assessing Mechanisms of Action. Handb Exp Pharmacol. 2018; 252:227-260).

Acute effects of psychedelics that may contribute to their therapeutic benefits include enhancing the therapeutic relationship by increased openness, trust, feelings of connectedness or emulsion with people, insight in psychological problems and stimulation of neuroregenerative processes as described in detail elsewhere (Vollenweider & Preller, 2020).

Psychedelic administration, especially at doses believed to be therapeutic, has a side effect of hallucinations or perceptual disturbances (Ungerleider, J. THOMAS. “The acute side effects from LSD.” The problems and prospects of LSD (1968): 61-68), (Nichols, Psychedelics, Pharmacol Rev 68: 264-355). These side effects make it unsafe for subjects to administer psychedelics except under direct medical supervision and could present a significant risk to patient safety if the side effects occur while the patient is driving, operating machinery or not under medical supervision. These side effects that are the hallmarks of psychedelic administration are mediated by the drug's activity at the serotonin 5-HT2A receptor, as evidenced by the fact that blockade of this activity (by administration of a 5-HT2A antagonist, such as ketanserin) can attenuate these psychoactive/hallucinogenic side effects (Holze et al., 2020).

Repeat administration of psychedelics (e.g., on a daily basis) has been shown to qualitatively have clinical benefit while the side effects of hallucination/perceptual disturbance may go away after a few days of treatment due to tachyphylaxis (i.e., via down regulation of 5-HT2A receptors) as reviewed by Buchborn (2016). Nonetheless, for the first days of administration of a psychedelic drug, the subject is likely to experience the common adverse events of hallucination, perceptual disturbance and others that may present a risk to the patient.

The Cleveland Clinic describes that titration is a method that limits potential side effects by taking time to see how one's body reacts to a drug, wherein medication is started at a low dose and then the dose is raised every couple of weeks until the maximum effective dose (target dose) has been achieved or side effects occur. Caffrey, et al. (Ther Adv Drug Saf. 2021 Jan. 19; 11: 2042098620958910) describes that titration is commonly used for drugs with a narrow therapeutic index to provide treatment at the lowest dose possible while minimizing medication use and side effects. It is a patient-centered approach to provide individualized therapy. Titration has been used for antibiotics, anticoagulants, anticonvulsants, antidepressants, antidiabetics, antipsychotics, opioids, and stimulants.

There remains a need for treatments with psychedelics that avoid or reduce unwanted side effects.

### SUMMARY OF THE INVENTION

The present invention provides for a method of dosing a psychedelic that avoids the side effects of hallucinations and perceptual disturbances by administering the psychedelic to an individual in a titrating dosing regimen and reducing side effects of hallucinations and perceptual disturbances.

The present invention provides for a kit for administering a titrating dosing regimen of a psychedelic, including a pharmaceutically effective amount of the psychedelic in dosage forms separated in packaging according to dose and time of administration in a titrating dosing regimen, and instructions for use.

The present invention also provides for a method of treating an individual with psychedelics, by administering the psychedelic to the individual having a condition or disease in a titrating dosing regimen and reducing side effects of hallucinations and perceptual disturbances during treatment.

### DESCRIPTION OF THE DRAWINGS

Other advantages of the present invention are readily appreciated as the same becomes better understood by reference to the following detailed description when considered in connection with the accompanying drawings wherein:

FIG. 1 is a graph of dose versus time.

### DETAILED DESCRIPTION OF THE INVENTION

The present invention provides for a method of dosing a psychedelic that avoids the side effects of hallucinations and perceptual disturbances by administering the psychedelic to an individual in a titrating dosing regimen and reducing side effects of hallucinations, perceptual disturbances, and other immediately detectable effects of the psychedelic while preserving desired therapeutic benefits.

More specifically, the titrating dosing regimen can include administering a starting dose to the individual, and at a set amount of time, increasing the dose a set amount and administering the increased dose to the individual, and repeating these steps over a period of time that the individual is being treated and until a maximum desired dose is reached. The dosing regimen can generally be described by the following equation: Dose=X(starting dose)+Y(dose increase)*Z(period of time)

As opposed to the prior uses of repeat daily administration (of for example 100 μg LSD every day), the starting dose can be a sub-perceptual dose (e.g., 10 μg) and taper up over time in a regimen that would never have the hallucinatory side effect but would achieve an effective dose that would be perceptual/hallucinogenic if administered in the absence of the titration regimen (e.g., 30, 50, 100 or 200 μg as the target therapeutic dose). For example, the starting dose can be 10 μg, which is increased by 10 μg every (2, 3, 4, 5, 6 or 7 days). Other starting doses can be within the ranges described below. Other examples of dosing can be found in Buchborn (2016).

The time period can be hours, days, weeks, months, or years, or intervals of dose titration with intervals of non-dosing, where each dose titration period can lead to the ability to avoid hallucinations, perceptual, and other detectable effects of the psychedelic, while preserving or enhancing desired therapeutic effects.

The starting dose and increased dose levels can be administered once per day, twice per day or three times per day, and can be administered by a care giver, healthcare provider, or self administered by the patient with or without supervision.

The dose increase can be any small amount such as 10, 20, 30 or 50 μg, and can be effected and determined by drug and formulation variation, and patient specific factors such as weight, height, body surface area, biochemical assays, metabolic assays, or genomic assays.

The dosage form used can be any suitable dosage form such as tablets, capsules, lozenges, transdermal patches, implanted devices, solutions, gels, emulsions, or any solid or liquid form, or some combination of forms, as well as those further described below.

The starting dose can also be a larger loading dose administered under medical supervision followed by repeat sub-perceptual doses to maintain the treatment benefit while limiting side effects to only the first dose.

The psychedelics in the present invention can be, but are not limited to, lysergic acid diethylamide (LSD), psilocybin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, analogs thereof, or homologues thereof. Preferably, the dose of the psychedelic is one that provides a meaningful effect. A dose of 0.01-1 mg (10-1000 μg) can be used of LSD. Psilocybin can be dosed at 5-50 mg, mescaline can be dosed at 50-800 mg, 5-MeO-DMT can be dosed at 1-20 mg, DMT can be dosed at 20-100 mg, DOI can be dosed at 0.1-5 mg, and DOB can be dosed at 0.1-5 mg. Effects of the psychedelic drug can last 1-12 hours after administration, and the individual can be supervised by medical personnel such as a psychiatrist during this time. If lower doses are given, medical supervision can be unnecessary.

The present invention can carry out its clinical effect by down-regulating (or reducing the expression of) the serotonin 5-HT2A receptor or by another mechanism of action that reduces the hallucinogenic effects of a psychedelic drug over time.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants, and vehicles. The compounds can be administered orally, transcutaneously, subcutaneously or parenterally including intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours, days, weeks, or months.

When administering the compound of the present invention parenterally, it will generally be formulated in a sublingual or buccal dissolving tablet, dissolving film, intranasal powder, intranasal solution, inhaled powder, inhaled solution, transdermal patch, transdermal patch (with microneedles or other permeation enhancers) or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a kit for administering a titrating dosing regimen of a psychedelic, including a pharmaceutically effective amount of the psychedelic in dosage forms separated in packaging according to dose and time of administration in a titrating dosing regimen, and instructions for use. The starting dose and each additional increased dose can be in different colors and/or sizes for an individual to easily distinguish them. The increased dose can be a single dosage form or multiple separate dosage forms (i.e., one dosage form (i.e., tablet, patch, etc.) for each dose increase). The packaging can indicate which time period each dose should be taken in, such as in hours, days, weeks, months, or years. The packaging can be in a bubble/blister pack form, which allows the individual to pop out the exact dose needed for each administration.

The present invention also provides for a method of treating an individual with psychedelics, by administering the psychedelic to the individual having a condition or disease in a titrating dosing regimen and reducing side effects of hallucinations and perceptual disturbances during treatment.

The condition or disease being treated with the methods of the present invention can include, but is not limited to, anxiety disorders (including anxiety in advanced stage illness e.g. cancer, as well as generalized anxiety disorder), depression (including post partum depression, major depressive disorder and treatment-resistant depression), headache disorder (including cluster headaches and migraine headache), obsessive compulsive disorder (OCD), personality disorders (including conduct disorder), stress disorders (including adjustment disorders and post-traumatic stress disorder), drug disorders (including alcohol dependence, nicotine dependence, opioid dependence, cocaine dependence, methamphetamine dependence), other addictions (including gambling disorder, eating disorder, and body dysmorphic disorder), pain, neurodegenerative disorders (such as dementia, Alzheimer's Disease, Parkinson's Disease), movement disorders (such as essential tremor, tardive dyskinesia), autism spectrum disorder, eating disorders, or neurological disorders (such as stroke or traumatic brain injury).

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

The following is an example of dosing to achieve uptitration of LSD. This is also applicable to other psychedelics. TABLE 1 shows oral administration over different days and for different times of daily administration. The dose levels can translate into 1) a PK threshold and 2) a 5-HT2A expression threshold that can be tailored via other dosing regiments and formulations so that the dosing is done in a way that patients never get PK levels that match the (increasing) threshold for perceptual effects.

TABLE 1

| Scenario | Day 1 | 2 | 3 | 4 | 5 | 6 | 7-14 | 14-28 | 29-56 | 57-84+ |
|---|---|---|---|---|---|---|---|---|---|---|
| 1 (daily admin) | 10 | 10 | 30 | 30 | 50 | 50 | 100 | 200 | 200 | 200 |
| 2 (2×/daily admin) | 10 | 20 | 40 | 80 | 100 | 200 | 200 | 200 | 200 | 200 |
| 3 | 20 | 50 | 100 | 100 | 200 | 200 | 200 | 200 | 200 | 200 |

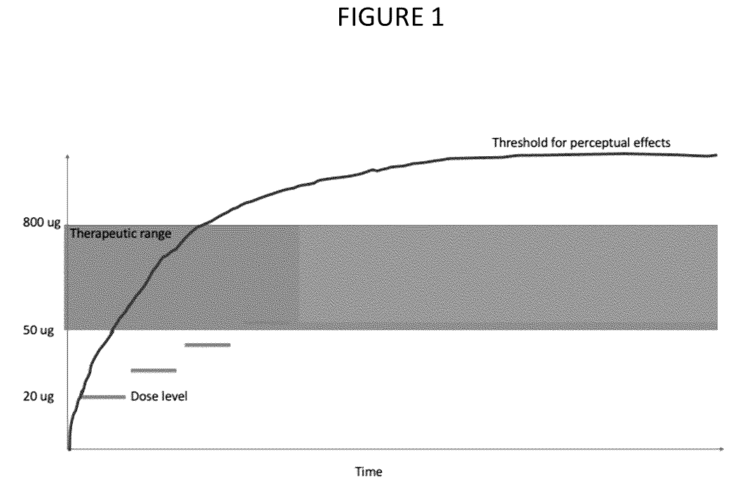

FIG. 1 shows how the dose increases over time to a threshold for perceptual effects. Similar effects would be present for PK levels. TABLE 2 shows dosing with an extended release formulation.

TABLE 2

| | | | | | | | | | | |
|---|---|---|---|---|---|---|---|---|---|---|
| Scenario | Day 1 | 2 | 3 | 4 | 5 | 6 | 7 | 14-28 | 29-56 | 57-84+ |

| | | | | |
|---|---|---|---|---|
| 1 | Patch #1 | Patch #2 | Patch #3 | Patch #3 (high dose) |
| | (small dose) | (mid dose) | (high dose) | weekly or monthly |
| 2 | Patch #1 | | Patch #2 | Patch #3 (high dose) |
| | (small dose) | | (mid dose) | weekly or monthly |
| 3 | Patch #2 | Patch #2 | Patch #2 | Patch #2 (mid dose) |
| | (mid dose) | (mid dose) | (mid dose) | weekly or monthly |

Precise packaging configurations can be used, such as a dosing kit of multiple patches that can be applied at regular intervals either at home or in a clinic. The kit design (i.e., specific way the patches are sized, any differences in their properties, and the actual packaging design) can ensure adherence and so that patients do not inadvertently expose themselves to a hallucinogenic dose.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of dosing a psychedelic that avoids the side effects of hallucinations and perceptual disturbances, including the steps of:
administering the psychedelic to an individual in a titrating dosing regimen; and
reducing side effects of hallucinations and perceptual disturbances.

2. The method of claim 1, wherein said administering step is further defined as:
administering a starting dose to the individual;
at a set amount of time, increasing the dose a set amount and administering the increased dose to the individual; and
repeating said increasing and administering steps over a period of time that the individual is being treated and until a maximum desired dose is reached.

3. The method of claim 2, wherein the starting dose is a sub-perceptual dose.

4. The method of claim 2, wherein the starting dose is 10 μg and is increased by 10 μg every period of time.

5. The method of claim 2, wherein the period of time is chosen from the group consisting of hours, days, weeks, months, and years.

6. The method of claim 2, wherein the dose is increased by an amount chosen from the group consisting of 10, 20, 30, and 50 μg.

7. The method of claim 1, wherein said administering step is further defined as administering a starting dose of a loading dose and administering subsequent doses of sub-perceptual doses.

8. The method of claim 1, wherein the psychedelic is chosen from the group consisting of lysergic acid diethylamide (LSD), psilocybin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, analogs thereof, and homologues thereof.

9. A kit for administering a titrating dosing regimen of a psychedelic, comprising a pharmaceutically effective amount of the psychedelic in dosage forms separated in packaging according to dose and time of administration in a titrating dosing regimen, and instructions for use.

10. The kit of claim 9, wherein said psychedelic is chosen from the group consisting of lysergic acid diethylamide (LSD), psilocybin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, analogs thereof, and homologues thereof.

11. The kit of claim 9, wherein said dosage forms include a starting dose and additional increased doses.

12. The kit of claim 11, wherein said starting dose is in a different color or size from said additional increased doses.

13. The kit of claim 11, wherein said additional increased doses are a single dosage form or multiple separate dosage forms.

14. The kit of claim 9, wherein said packaging indicates which time period each dose should be taken in.

15. The kit of claim 9, wherein said packaging is a blister pack.

16. A method of treating an individual with psychedelics, including the steps of:
administering the psychedelic to the individual having a condition or disease in a titrating dosing regimen; and
reducing side effects of hallucinations and perceptual disturbances during treatment.

17. The method of claim 16, wherein the condition or disease being treated is chosen from the group consisting of anxiety disorders, depression, headache disorder, obsessive compulsive disorder (OCD), personality disorders, stress disorders, drug disorders, gambling disorder, eating disorder, body dysmorphic disorder, pain, neurodegenerative disorders, movement disorders, autism spectrum disorder, eating disorders, and neurological disorders.

18. The method of claim 16, wherein said administering step is further defined as:
administering a starting dose to the individual;
at a set amount of time, increasing the dose a set amount and administering the increased dose to the individual; and
repeating said increasing and administering steps over a period of time that the individual is being treated and until a maximum desired dose is reached.

19. The method of claim 18, wherein the starting dose is a sub-perceptual dose.

20. The method of claim 18, wherein the starting dose is 10 μg and is increased by 10 μg every period of time.

21. The method of claim 18, wherein the period of time is chosen from the group consisting of hours, days, weeks, months, and years.

22. The method of claim 18, wherein the dose is increased by an amount chosen from the group consisting of 10, 20, 30, and 50 μg.

23. The method of claim 16, wherein said administering step is further defined as administering a starting dose of a loading dose and administering subsequent doses of sub-perceptual doses.

24. The method of claim 16, wherein the psychedelic is chosen from the group consisting of lysergic acid diethylamide (LSD), psilocybin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, tartrates thereof, analogs thereof, and homologues thereof.
